(19) United States
(12) Patent Application Publication
(10) Pub. No.: US 2002/0028793 A1
(43) Pub. Date: Mar. 7, 2002

(54) Use of estrogens and delta-gonadien-21-3,20-diones

(76) Inventor: Karsten Wassermann, Gentofte (DK)

Correspondence Address: Steve T Zelson Esq, Novo Nordisk of North America Inc, 405 Lexington Avenue, Suite 6400, New York, NY, 10174-6400, US

(21) Appl. No.: 09/484,673

(22) Filed: Jan. 18, 2000

(30) Foreign Application Priority Data
Jan. 18, 1999 (DK) 1999 00053

(51) Int. Cl. A61K 31/56

(52) U.S. Cl. 514/171; 514/824

(57) ABSTRACT

The present invention relates to the use of estrogens and delta-gonadien-21-ol-3,20-diones in the treatment or prophylaxis of hyperlipoproteinemia, hypertriglyceridemia, hyperlipidemia or hypercholesterolemia or arteriosclerosis or for anticoagulative treatment.

### CROSS-REFERENCE TO RELATED APPLICATIONS

This application claims priority under 35 U.S.C. 119 of Danish application No. 1999 00053 filed Jan. 18, 1999, the contents of which are fully incorporated herein by reference.

### BACKGROUND OF THE INVENTION

1. Field of the Invention

The present invention relates to the use of a combination of estrogens or SERMs (selective estrogen receptor modulators) with delta-gonadien-21-ol-3,20-diones for the treatment of patients suffering from hyperlipoproteinemia, hypertriglyceridemia, hyperlipidemia or hypercholesterolemia and prophylaxis thereof. The present invention furthermore relates to the use of a combination of estrogens or SERMs with delta-gonadien-21-ol-3,20-diones for the treatment of patients suffering from arteriosclerosis including atherosclerosis and prophylaxis thereof, and furthermore to the use of a combination of estrogens or SERMs with delta-gonadien-21-ol-3,20-diones for the treatment of patients in need of an anticoagulative treatment or prophylaxis, e.g., following a coronary thrombosis or postoperatively, i.e., after surgery. The present invention also embraces pharmaceutical compositions and kits comprising these compounds and methods of using the compounds and their pharmaceutical compositions.

2. Description of the Related Art

lschemic heart disease (IHD), the relevant cardiovascular disease in relation to postmenopausal women, primarily is caused by atherosclerosis (Havel and Rapaport, N Eng J Med 1995; 332:1491-1498). Other frequent manifestations of atherosclerosis are cerebrovascular disease, and intermittent claudication. An important risk factor for the development of atherosclerosis is an atherogenic lipid profile, i.e., hyperlipidemia with increased LDL-cholesterol and relatively decreased HDL-cholesterol. In epidemiological studies (Samsioe G. Int J Fertil, 1993;38, suppl. 1:19-23) it has been indicated that estrogen therapy in post-menopausal women reduces the stenosis of the coronary arteries, thereby increasing survival rate compared to a non-treated population. An important factor in this effect on the coronary system is a reduction in serum lipids and a normalization of the relation between LDL-cholesterol and HDL-cholesterol (Samsioe G. Int J Fertil 1994; 39 suppl. 1:43-49).

One object of the present invention is to provide compositions which can effectively be used in the treatment or prophylaxis of hyperlipoproteinemia, hypertriglyceridemia, hyperlipidemia or hypercholesterolemia.

Data on men suggest that a 35% reduction in LDL-cholesterol is required to achieve a 50% reduction in cardiovascular disease, i.e., coronary atherosclerosis provided that LDL-cholesterol reduction is the sole cause of cardioprotection. However, estrogens reduce LDL-cholesterol by only 5-10%. It is therefore now believed that the lipid effects account for only 25-40% of the reduction in the incidence of coronary heart disease after estrogen replacement therapy. A possible mechanism could be a direct effect on the vessel wall improving blood flow and inhibiting the atherogenic mechanisms independent on an effect on plasma lipids.

Another object of the present invention is to provide compositions which can effectively be used in the treatment or prophylaxis of arteriosclerosis including atherosclerosis.

Coagulation and thrombosis are important mechanisms involved in the pathogenesis of atherosclerosis and its complications such as vascular occlusion and embolism. Furthermore, blood clotting is important for the development of vascular restenosis following surgical intervention of blocked arteries. Restenosis occurs in about 35% of the patients after 6 months. Current therapy such as heparin, low molecular heparin and aspirin or stents have failed to reduce the incidence of restenosis. New therapeutic possibilities which can inhibit a tendency for thrombosis after endothelial damage are therefore needed.

Another further object of the present invention is to provide compositions which can effectively be used in the treatment or prophylaxis of patients in need of an anticoagulative treatment or prophylaxis, e.g., following a coronary thrombosis or after surgery.

There remains a need in the art for combined compositions and methods which have beneficial effects on hyperlipoproteinemia, hypertriglyceridemia, hyperlipidemia or hypercholesterolemia or arteriosclerosis including atherosclerosis or as an anticoagulant, but without introducing significant effects in the reproductive tissues. There is a further need for such compositions that lack or have decreased undesirable side effects of estrogen(s) and/or progesterone(s).

One object of the present invention is to provide compositions in one dosage form which can effectively be used in the treatment or prophylaxis of hyperlipoproteinemia, hypertriglyceridemia, hyperlipidemia or hypercholesterolemia or arteriosclerosis including atherosclerosis or as an anticoagulant.

Another object of the present invention is to provide compositions, method of treatment or kits exhibiting a synergistic effect.

A further object of the present invention is to provide compositions, method of treatment or kits exhibiting no substantial side effects, such as high level of coronary heart disease events.

Other objects of the present invention will become apparent upon reading the present description.

### BRIEF DESCRIPTION OF THE DRAWING

FIG. 1 shows graphs analyzing full-blood glucose, serum triglycerides and total serum cholesterol in the Type 2 diabetic mouse model db/db.

### DESCRIPTION OF THIS INVENTION

The present invention is based in part on the discovery that a representative combination of an estrogen or estrogen receptor modulator and a compound of formula I

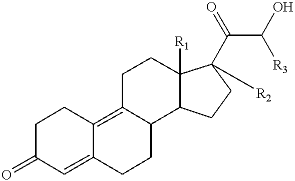

wherein R₁, R₂ and R₃ independently of each other are C₁₋₁₂alkyl, in the form of 21R or 21S epimers or mixtures thereof, or a pharmaceutically acceptable salt thereof, is effective as a hypolipoproteinemic, hypotriglyceridemic, hypolipidemic or hypocholesterolemic composition. The data indicate that the present compositions are useful as therapeutic and preventive hypolipoproteinemic, hypotriglyceridemic, hypolipidemic or hypocholesterolemic agents in mammals, including primates such as humans.

Furthermore, the present invention is based in part on the discovery that a representative combination of an estrogen or estrogen receptor modulator and a compound of formula I is also effective against the direct vascular effects of arteriosclerosis including atherosclerosis. The data indicate that the combination is useful as a therapeutic agent against arteriosclerosis including atherosclerosis in mammals, including primates such as humans.

The present invention is yet further based in part on the discovery that a representative combination of an estrogen or estrogen receptor modulator and a compound of formula I has preventive or therapeutic anticoagulative activities when administered inter alia to rats or mice. The data indicate that the combination is useful as a therapeutic and preventive agent to mammals, including primates such as humans, in need of an anticoagulative treatment or prophylaxis, e.g., following a coronary thrombosis or after surgery.

This invention is related to the treatment or prophylaxis of disorders as defined in the Lipid Research Clinics Program. J.A.M.A. 251 (1984), 351-364 and J.A.M.A. 251 (1984), 365-374 or what a person skilled in the art may consider as subject for treatment or prophylaxis.

The combination of an estrogen or estrogen receptor modulator and a compound of formula I shows a synergistic effect in treatment of hyperlipoproteinemia, hypertriglyceridemia, hyperlipidemia or hypercholesterolemia or arteriosclerosis or for anticoagulative treatment.

Moreover, the combination of an estrogen or estrogen receptor modulator and a compound of formula I shows a synergistic effect in lowering total serum cholesterol. Furthermore, the combination of an estrogen or estrogen receptor modulator and a compound of formula I shows a synergistic effect in lowering triglycerides.

In a first aspect the invention relates to a method of treating hyperlipoproteinemia, hypertriglyceridemia, hyperlipidemia or hypercholesterolemia, which method comprises administering to a subject an effective amount of an estrogen or estrogen receptor modulator in combination with an effective amount of a compound of formula I

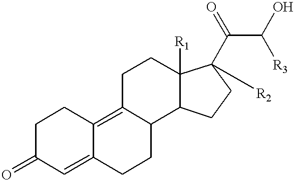

wherein R₁, R₂ and R₃ independently of each other are C₁₋₁₂alkyl, in the form of 21R or 21S epimers or mixtures thereof, or a pharmaceutically acceptable salt thereof, in an amount sufficient to treat hyperlipoproteinemia, hypertriglyceridemia, hyperlipidemia or hypercholesterolemia.

In one embodiment of the first aspect it is a method of treating hyperlipoproteinemia. In a second embodiment of the first aspect it is a method of treating hypertriglyceridemia. In a third embodiment of the first aspect it is a method of treating hyperlipidemia. In a fourth embodiment of the first aspect it is a method of treating hypercholesterolemia.

In a second aspect the invention relates to a method of treating arteriosclerosis including atherosclerosis, which method comprises administering to a subject an effective amount of an estrogen or estrogen receptor modulator in combination with an effective amount of a compound of formula I

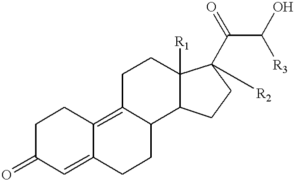

wherein R₁, R₂ and R₃ independently of each other are C₁₋₁₂alkyl, in the form of 21R or 21S epimers or mixtures thereof, or a pharmaceutically acceptable salt thereof, in an amount sufficient to treat arteriosclerosis including atherosclerosis.

In a third aspect the invention relates to a method for the treatment of patients in need of an anticoagulative treatment, e.g., following a coronary thrombosis or after surgery, which method comprises administering to a subject an effective amount of an estrogen or estrogen receptor modulator in combination with an effective amount of a compound of formula I

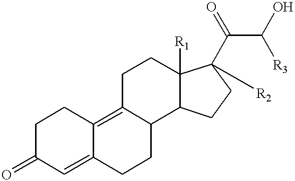

wherein R₁, R₂ and R₃ independently of each other are C₁₋₁₂alkyl, in the form of 21R or 21S epimers or mixtures thereof, or a pharmaceutically acceptable salt thereof, in an amount sufficient for the treatment of patients in need of an anticoagulative treatment, e.g,. following a coronary thrombosis or after surgery.

In a fourth aspect the invention relates to a kit containing a treatment for hyperlipoproteinemia, hypertriglyceridemia, hyperlipidemia or hypercholesterolemia or arteriosclerosis or for anticoagulative treatment, comprising: a) an effective amount of an estrogen or estrogen receptor modulator and a pharmaceutically acceptable carrier in a first unit dosage form; b) an effective amount of a compound of formula I

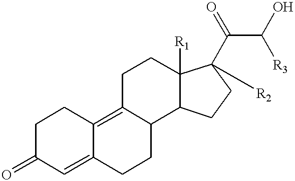

wherein R₁, R₂ and R₃ independently of each other are C₁₋₁₂alkyl, in the form of 21R or 21S epimers or mixtures thereof, or a pharmaceutically acceptable salt thereof, and a pharmaceutically acceptable carrier in a second unit dosage form; and c) means for containing said first and second dosage forms. Each of said treatments is considered an individual embodiment of the invention.

In a fifth aspect the invention relates to the use of an estrogen or estrogen receptor modulator in combination with an effective amount of a compound of formula I

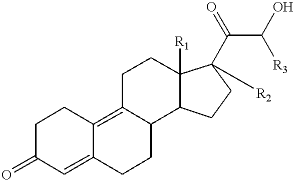

wherein R₁, R₂ and R₃ independently of each other are C₁₋₁₂alkyl, in the form of 21R or 21S epimers or mixtures thereof, or a pharmaceutically acceptable salt thereof, for the manufacture of a medicament for treating hyperlipoproteinemia, hypertriglyceridemia, hyperlipidemia or hypercholesterolemia or arteriosclerosis or for anticoagulative treatment. Each of said treatments is considered an individual embodiment of the invention.

In a further aspect the invention relates to a method of lowering total serum cholesterol, which method comprises administering to a subject an effective amount of an estrogen or estrogen receptor modulator in combination with an effective amount of a compound of formula I.

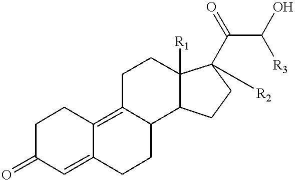

wherein R₁, R₂ and R₃ independently of each other are C₁₋₁₂alkyl, in the form of 21R or 21S epimers or mixtures thereof, or a pharmaceutically acceptable salt thereof, in an amount sufficient to lower total serum cholesterol.

In a further aspect the invention relates to a method of lowering triglycerides, which method comprises administering to a subject an effective amount of an estrogen or estrogen receptor modulator in combination with an effective amount of a compound of formula I

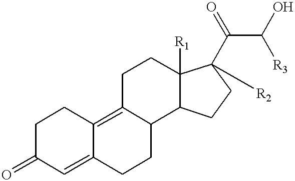

wherein R₁, R₂ and R₃ independently of each other are C₁₋₁₂alkyl, in the form of 21R or 21S epimers or mixtures thereof, or a pharmaceutically acceptable salt thereof, in an amount sufficient to lower triglycerides.

In a further aspect the invention relates to a kit containing a treatment for lowering total serum cholesterol, comprising: a) an effective amount of an estrogen or estrogen receptor modulator and a pharmaceutically acceptable carrier in a first unit dosage form; b) an effective amount of a compound of formula I

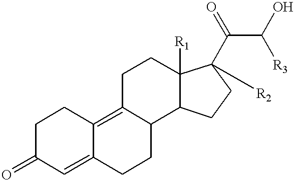

wherein R₁, R₂ and R₃ independently of each other are C₁₋₁₂alkyl, in the form of 21R or 21S epimers or mixtures thereof, or a pharmaceutically acceptable salt thereof, and a pharmaceutically acceptable carrier in a second unit dosage form; and c) means for containing said first and second dosage forms.

In a further aspect the invention relates to a kit containing a treatment for lowering triglyceride,s comprising: a) an effective amount of an estrogen or estrogen receptor modulator and a pharmaceutically acceptable carrier in a first unit dosage form; b) an effective amount of a compound of formula I

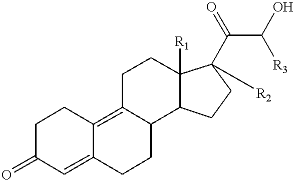

wherein R₁, R₂ and R₃ independently of each other are C₁₋₁₂alkyl, in the form of 21R or 21S epimers or mixtures thereof, or a pharmaceutically acceptable salt thereof, and a pharmaceutically acceptable carrier in a second unit dosage form; and c) means for containing said first and second dosage forms.

In a further aspect the invention relates to a use of an estrogen or estrogen receptor modulator in combination with an effective amount of a compound of formula I

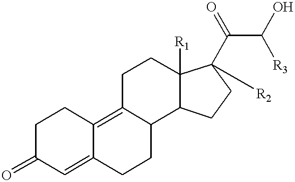

wherein R₁, R₂ and R₃ independently of each other are C₁₋₁₂alkyl, in the form of 21R or 21S epimers or mixtures thereof, or a pharmaceutically acceptable salt thereof, for the manufacture of a medicament for lowering total serum cholesterol.

In a further aspect the invention relates to the use of an estrogen or estrogen receptor modulator in combination with an effective amount of a compound of formula I

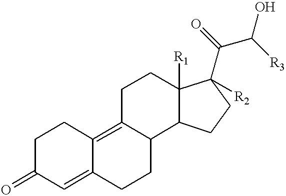

wherein R₁, R₂ and R₃ independently of each other are C₁₋₁₂alkyl, in the form of 21R or 21S epimers or mixtures thereof, or a pharmaceutically acceptable salt thereof, for the manufacture of a medicament for lowering triglycerides.

In a further aspect the invention relates to a composition, such as a pharmaceutical composition, comprising an estrogen or estrogen receptor modulator and a compound of formula I

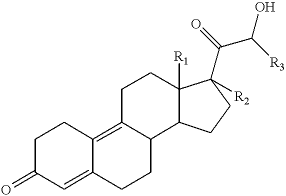

wherein R₁, R₂ and R₃ independently of each other are C₁₋₁₂alkyl, in the form of 21R or 21S epimers or mixtures thereof, or a pharmaceutically acceptable salt thereof, and a pharmaceutically acceptable carrier.

In one embodiment of the present invention the estrogen or estrogen receptor modulator and the compound of formula I is administered simultaneously in one dosage form, preferably orally as a tablet or capsule or as a transdermal patch.

In another embodiment of the present invention the estrogen or estrogen receptor modulator and the compound of formula I is administered substantially simultaneously.

In a further embodiment of the present invention R₁, R₂ and R₃ are independently of each other a C₁₋₆alkyl, such as C₁₋₄alkyl, preferably methyl.

In a further preferred embodiment of the present invention the compound of formula I is selected from

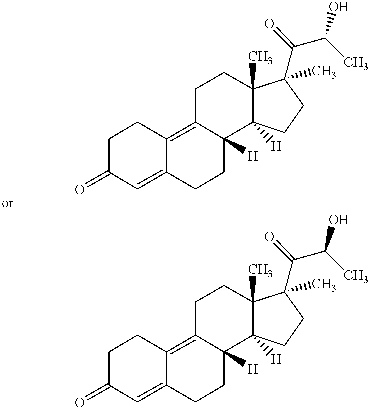

In a preferred embodiment of the present invention the compound of formula I is

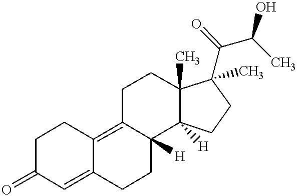

In a still further embodiment of the present invention the estrogen is selected from 17-beta-estradiol and esters thereof, ethinylestradiol, estriol (trihydroxyestrin), estrone, conjugated estrogens (eg. Premarin), sodium estrone sulfate, 8(9)-dehydroestradiol derivatives, 17alfa-dihydroequilin, equilenin, 17alfa-dihydroequilenin, esterified estrogens, and equilin, preferably 17-beta-estradiol and esters thereof, ethinylestradiol and conjugated estrogens. Each of these estrogens is individually considered an embodiment of the invention.

In a further embodiment of the present invention the estrogen receptor modulator is selected from droloxifene, raloxifene, tamoxifen, 4-hydroxy-tamoxifen, idoxifene, centchroman, levormeloxifene, Cis-6-(4-fluoro-phenyl)-5-[4-2-piperidin-1-yl-ethoxy)-phenyl]-5,6,7,8-tetrahydronaphthalene-2-ol; (−)-Cis-6-phenyl-5-[4-(2-pyrrolidin-1-yl-ethoxy)-phenyl]-5,6,7,8-tetrahydronaphthalene -2-ol; Cis-6-phenyl-5-[4-(2-pyrrolidin-1-yl-ethoxy)-phenyl]-5,6,7,8-tetrahydronaphthalene-2-ol; Cis-1-[6′-pyrrolidinoethoxy-3′-pyridyl]-2-phenyl-6-hydroxy-1,2,3,4-tetrahydronaphthalene; 1-(4′-Pyrrolidinoethoxyphenyl)-2-(4′-fluorophenyl)-6-hydroxy-1,2,3,4-tetrahydroisoquinoline; Cis-6-(4-hydroxyphenyl)-5-[4-(2-piperidin-1-yl-ethoxy)-phenyl]-5,6,7,8-tetrahydronaphthalene-2-ol; and 1-(4′-Pyrrolidinoethoxyphenyl)-2-phenyl-6-hydroxy-1,2,3,4-tetrahydroisoquinoline. Each of these modulators is individually considered an embodiment of the invention.

In a still further embodiment of the present invention the effective amount of an estrogen or estrogen receptor modulator is from 0.00001 to 1000 mg/day, such as 0.01 to 2.5 mg/day and the effective amount of a compound of formula I is from 0.00001 to 1000 mg/day, such as 0.01 to 1.0 mg/day.

Within the present invention, the estrogen or estrogen receptor modulator and the compound of formula I may be prepared in the form of pharmaceutically acceptable salts, especially acid-addition salts, including salts of organic acids and mineral acids. Examples of such salts include salts of organic acids such as formic acid, fumaric acid, acetic acid, propionic acid, glycolic acid, lactic acid, pyruvic acid, oxalic acid, succinic acid, malic acid, tartaric acid, citric acid, benzoic acid, salicylic acid and the like. Suitable inorganic acid-addition salts include salts of hydrochloric, hydrobromic, sulphuric and phosphoric acids and the like. Each of these salts is individually considered an embodiment of the invention.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The C₁₋₁₂-alkyl, C₁₋₆-alkyl or C₁₋₄-alkyl groups specified above are intended to include those alkyl or alkylene groups of the designated length in either a linear or branched or cyclic configuration. Examples of linear alkyl are methyl, ethyl, propyl, butyl, pentyl, and hexyl and their corresponding divalent moieties, such as ethylene. Examples of branched alkyl are isopropyl, sec-butyl, tert-butyl, isopentyl, and isohexyl and their corresponding divalent moieties, such as isopropylene. Examples of cyclic alkyl are C₃₋₆-cycloalkyl such as cyclopropyl, cyclobutyl, cyclopentyl and cyclohexyl and their corresponding divalent moieties, such as cyclopropylene.

The compositions and kits of the present invention are useful within human and veterinary medicine, for example, in the treatment or prophylaxis of patients suffering from hyperlipoproteinemia, hypertriglyceridemia, hyperlipidemia or hypercholesterolemia or arteriosclerosis or for anticoagulative treatment. For use within the present invention, the estrogens or estrogen receptor modulators and compounds of formula I and their pharmaceutically acceptable salts are formulated with a pharmaceutically acceptable carrier to provide a medicament for parenteral, oral, nasal, rectal, subdermal, intradermal or transdermal administration according to conventional methods. Formulations may further include one or more diluents, fillers, emulsifiers, preservatives, buffers, excipients, etc. and may be provided in such forms as liquids, powders, emulsions, suppositories, liposomes, transdermal patches, controlled release, dermal implants, tablets, etc. One skilled in this art may formulate the compounds of formula I in an appropriate manner, and in accordance with accepted practices, such as those disclosed in Remington's Pharmaceutical Sciences, Gennaro, ed., Mack Publishing Co., Easton, Pa., 1990.

Oral administration is preferred. Thus, the estrogen or estrogen receptor modulator and compound of formula I are prepared in a form suitable for oral administration, such as a tablet or capsule, that is either a tablet or capsule containing both the estrogen or estrogen receptor modulator and compound of formula I in one dosage form, or a tablet or capsule containing the estrogen or estrogen receptor modulator in one dosage form and a tablet or capsule containing the compound of formula I in another dosage form. Typically, a pharmaceutically acceptable salt of the compound of formula I is combined with a carrier and molded into a tablet. Suitable carriers in this regard include starch, sugars, dicalcium phosphate, calcium stearate, magnesium stearate and the like. Such compositions may further include one or more auxiliary substances, such as wetting agents, emulsifiers, preservatives, stabilizers, coloring additives, etc.

Pharmaceutical compositions containing the estrogen or estrogen receptor modulator and the compound of formula I may be administered one or more times per day or week. An effective amount of such a pharmaceutical composition is the amount that provides a clinically significant effect against hyperlipoproteinemia, hypertriglyceridemia, hyperlipidemia or hypercholesterolemia or arteriosclerosis or for anticoagulative patients. Such amounts will depend, in part, on the particular condition to be treated, age, weight, and general health of the patient, and other factors evident to those skilled in the art.

A typical oral dose will contain a nontoxic dosage range of from about 0.0001 to about 100 mg/kg patient per day of the estrogen or estrogen receptor modulator. A suitable oral dose of a compound of formula I is from 0.0001 to 100 mg/kg patient per day. In sequential regimen oral forms preferred dosages are from 0.001 to 50 mg, in particular 0.01 to 10 mg, more preferred 0.05 to 5 mg, most preferred 0.3 to 2.0 mg estrogen, e.g., 17-beta-estradiol or conjugated equine estrogens, continuously combined with 10-14 days of 0.0001 to 10 mg, preferred 0.001 to 10 mg, in particular 0.01 to 5 mg, most preferred 0.05 to 0.5 mg of compound of formula I, e.g., a compound of formula II. In continuous regimen oral forms preferred dosages are from 0.001 to 50 mg, in particular 0.01 to 10 mg, more preferred 0.05 to 5 mg, most preferred 0.3 to 2.0 mg estrogen, e.g., 17-beta-estradiol or conjugated equine estrogens, continuously combined with 0.0001 to 10 mg, preferred 0.001 to 10 mg, in particular 0.01 to 5 mg, most preferred 0.05 to 0.5 mg of compound of formula I, e.g., a compound of formula II, continuously.

A typical transdermal dose will contain a nontoxic dosage range of from about 0.00001 to about 100 mg/kg patient per day of the estrogen or estrogen receptor modulator. A suitable transdermal dose of a compound of formula I is from 0.00001 to 100 mg/kg patient per day. In sequential regimen transdermal forms preferred dosages are from 0.0001 to 50 mg, in particular 0.001 to 1 mg, more preferred 0.01 to 0.5 mg, most preferred 0.02 to 0.1 mg estrogen, eg. 17-beta-estradiol or conjugated equine estrogens, continuously combined with 10-14 days of 0.0001 to 10 mg, preferred 0.001 to 1 mg, in particular 0.01 to 0.5 mg, most preferred 0.05 to 0.4 mg of compound of formula I, e.g,. a compound of formula II. In continuous regimen transdermal forms preferred dosages are from 0.0001 to 50 mg, in particular 0.001 to I mg, more preferred 0.01 to 0.5 mg, most preferred 0.02 to 0.1 mg estrogen, eg. 17-beta-estradiol or conjugated equine estrogens, continuously combined with 0.0001 to 10 mg, preferred 0.001 to 1 mg, in particular 0.01 to 0.5 mg, most preferred 0.05 to 0.4 mg of compound of formula I, eg. a compound of formula II, continuously.

Subject or patient is intended to mean mammals, in particular humans, such as women in the menopause or postmenopausal women.

Treatment as used herein is intended to include prophylactic treatment and palliative treatment.

The pharmaceutical compositions containing an estrogen or estrogen receptor modulator and a compound of formula I may be administered in unit dosage form one or more times per day or week. In the alternative, they may be provided as controlled release formulations suitable for dermal implantation. Implants are formulated to provide release of active compound over the desired period of time, which can be up to several years. Controlled-release formulations are disclosed by, for example, Sanders et al., J. Pharm. Sci. 73 (1964), 1294-1297, 1984. Controlled-release formulations are also disclosed by U.S. Pat. No. 4,489,056 and U.S. Pat. No. 4,210,644, which are incorporated herein by reference.

Since the present invention relates to the prevention or treatment of hyperlipoproteinemia, hypertriglyceridemia, hyperlipidemia or hypercholesterolemia or arteriosclerosis or for anti-coagulative treatment by treatment with a combination of active ingredients which may be administered separately, the invention also relates to combining separate pharmaceutical compositions in kit form. The kit includes two separate pharmaceutical compositions: in one embodiment an estrogen and a delta4,9-gonadiene-21-ol-3,20-dione of formula I; and in another embodiment an estrogen receptor modulator and a delta4,9-gonadiene-21-ol-3,20-dione of formula I. The kit includes means for containing the separate compositions such as a divided bottle or a divided foil packet. Typically the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well-known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

The term “estrogen” or “estrogens” has its conventional meaning and comprises estrogen and estrogen derivatives such as 17-beta-estradiol and esters thereof, ethinylestradiol, estriol (trihydroxyestrin), estrone, conjugated estrogens, e.g., disclosed in U.S. Pat. Nos. 2,720,483 and 2,565,115, in particular premarin cf. internet place www.equinerescue.org/pmu_link.html, sodium estrone sulfate, 8(9)-dehydroestradiol derivatives as disclosed in WO 98/16544, 17alpha-dihydroequilin, equilenin, 17alpha-dihydroequilenin, esterified estrogens, and equilin.

Selective estrogen receptor modulators (SERMs), which previously were characterized as estrogen antagonists/partial agonists on their basis of their binding to the estrogen receptor alpha, act as full estrogen agonists in bone. The acronym SERM takes into account the fact that the activity of these agents is tissue selective and they cannot be definitely labeled as agonsists or antagonists but only as modulators of the estrogen receptor until their actions in specific tissues have been evaluated (Gustafsson, Current Opin Chem Biol 1998; 2:508-511). Thus, the term “SERM”s or “estrogen receptor modulators” has its conventional meaning and comprises droloxifene, raloxifene, tamoxifen, 4-hydroxy-tamoxifen, idoxifene, centchroman, Cis-6-(4-fluoro-phenyl)-5-[4-2-piperidin-1 -yl-ethoxy)-phenyl]-5,6,7,8-tetrahydronaphthalene-2-ol; (−)-Cis-6-phenyl-5-[4-(2-pyrrolidin-1-yl-ethoxy)-phenyl]-5,6,7,8-tetrahydronaphthalene -2-ol; Cis-6-phenyl-5-[4-(2-pyrrolidin-1-yl-ethoxy)-phenyl]-5,6,7,8-tetrahydronaphthalene-2-ol; Cis-1-[6′-pyrrolidinoethoxy-3′-pyridyl]-2-phenyl-6-hydroxy-1,2,3,4-tetrahydronaphthalene; 1-(4′-Pyrrolidinoethoxyphenyl)-2-(4′-fluorophenyl)-6-hydroxy-1,2,3,4-tetrahydroisoquinoline; Cis-6-(4-hydroxyphenyl)-5-[4-(2-piperidin-1-yl-ethoxy)-phenyl]-5,6,7,8-tetrahydronaphthalene-2-ol; 1-(4′-Pyrrolidinoethoxyphenyl)-2-phenyl-6-hydroxy-1,2,3,4-tetrahydroisoquinoline, and the like.

The present invention is further illustrated by the following examples which, however, are not to be construed as limiting the scope of protection. The chemist of ordinary skill in the art will recognize that certain compounds related to this invention will contain one or more atoms which may be in a particular stereochemical or geometric configuration, giving rise to stereoisomers and configurational isomers. All such isomers and mixtures thereof are included within the present invention. Some of the compounds related to the present invention have asymetric carbon atoms and are enantiomers or diastereomers. Diastereomeric mixtures can be separated into their individual diastereomers on the basis of their physical chemical differences by methods known per se. Such methods may be chromatography and/or fractional crystallization. Enantiomers can be separated by converting the enantiomeric mixture into a diastereomeric mixture by reaction with an appropriate optically active compound, e.g., alcohol, separating the diastereomers and converting, e.g., hydrolyzing, the individual diastereomers to the corresponding pure enantiomers. All such isomers, including diastereomers, enantiomers and mixtures thereof are considered a part of this invention.

The features disclosed in the foregoing description and in the following examples may, both separately and in any combination thereof, be material for realizing the invention in diverse forms thereof.

### EXAMPLES

Conjugated estrogens may be obtained following the process described in U.S. Pat. Nos. 2,720,483 or 2,565,115, which are incorporated herein by reference.

The compound having formula II

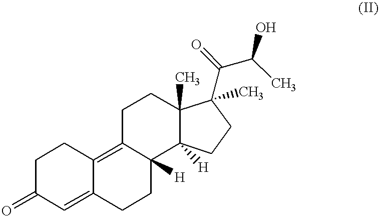

may be prepared as described in U.S. Pat. No. 4,273,771. The compound of formula II is called trimegestone.

An example of a tablet contains a conjugated estrogen (0.6 mg) and a compound of formula II (2.4 mg) formulated with pharmaceutically acceptable carriers to provide a medicament for oral administration according to conventional methods. The formulation further includes the following diluents, fillers, emulsifiers, preservatives, buffers and/or excipients, that is calcium phosphate tribasic, calcium sulfate, canauba wax, cellulose, glyceryl monooleate, lactose, magnesium stearate, methylcellulose, pharmaceutical glaze, polyethylene glycol, sucrose, povidone, titanium dioxide and red ferric oxide. One skilled in this art may formulate the tablet composition in an appropriate manner, and in accordance with accepted practices, such as those disclosed in Remington's Pharmaceutical Sciences, Gennaro, ed., Mack Publishing Co., Easton, Pa., 1990.

### Assessment of Pharmacological Activity in the Type 2 Diabetic Mouse Model db/db

Animals:

Male db/db mice were used as a model for type 2 diabetes. The db mouse is of C57BL/KsBom background and has the mutation localized to chromosome 4. The homozygotic db/db mouse is characterized by obesity, hyperphagia, hyperinsulinemia and hyperglycemia. As with most other conditions of hyperinsulinemia, the insulin response to glucose eventually becomes impaired leading ultimately to severe glucose intolerance. Due to these phenotypic characteristics this mouse model is recognized as a model of type 2 diabetes. The animals used in these studies were 13 weeks of age and at a time point of hyperinsulinemia and severe hyperglycemia with modest hypertriglyceridemia. All animal procedures were conducted according to Novo Nordisk A/S Animal Care approved protocols, and the experiments were done in compliance with internal animal welfare and national guidelines.

The animals were allowed to adapt to the laboratory conditions for 2 weeks prior to the experimental procedure. Normal chow and tap water were freely available in the home cages throughout the studies. A normal 12 h/12 h light/dark regime was operative (lights on at 06.00 hours) and room temperature was held between 20-23° C.

Experimental Procedure:

Animals were allocated to respective groups of treatment at the age of 13 weeks and with 6 animals per group. Full-blood glucose (non-fasting) was measured prior to treatment. Different groups of animals were injected SC daily with 17beta-estradiol valerate (0.03 mg/kg), trimegestone (0.30 mg/kg), a combination of 17beta-estradiol valerate (0.03 mg/kg) and trimegestone (0.30 mg/kg), or with the vehicle only (peanut oil). After 7 days of treatment full-blood glucose, serum triglycerides and total serum cholesterol were measured from samples of blood drawn from the retro-orbital sinus in non-fasting animals. An oral glucose tolerance test was performed on day 9 after an overnight fasting. Blood was sampled from the tail vein at time 0 min (baseline) and at 30, 60 and 120 min upon an oral glucose load of 3 g glucose/kg.

Analysis:

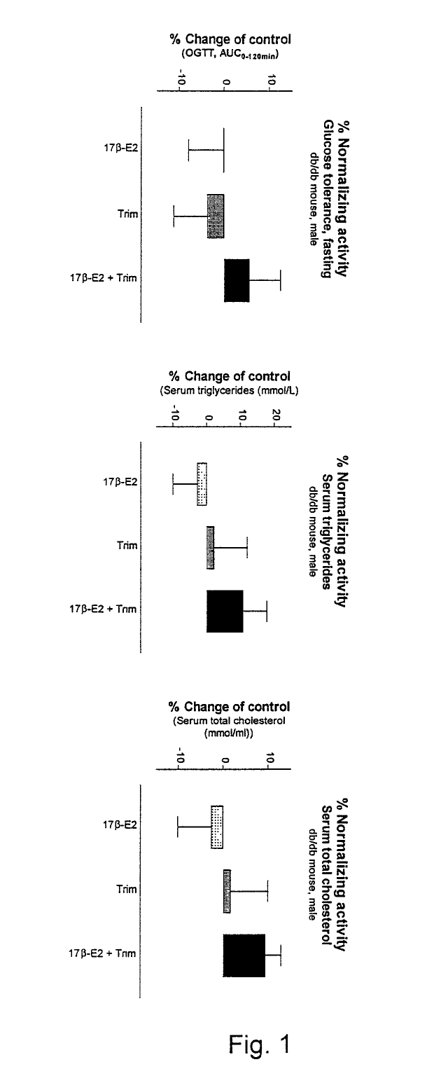

Analysis of full-blood glucose, serum triglycerides and total serum cholesterol were performed. Glucose levels in blood samples from the oral glucose tolerance test were used for calculation of the incremental Area Under the Curve (AUC_{0-120min-baseline}). All data are expressed as percentage change of vehicle treated animals (cf. FIG. 1).

What is claimed is:

1. A method of lowering total serum cholesterol, which method comprises administering to a subject an effective amount of an estrogen or estrogen receptor modulator in combination with an effective amount of a compound of formula I 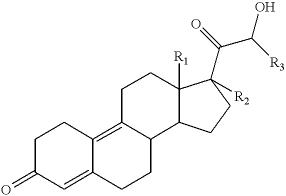
wherein R₁, R₂ and R₃ independently of each other are C₁₋₁₂alkyl, in the form of 21R or 21S epimers or mixtures thereof, or a pharmaceutically acceptable salt thereof, the effective amounts being sufficient to lower total serum cholesterol.

2. A method of lowering triglycerides, which method comprises administering to a subject an effective amount of an estrogen or estrogen receptor modulator in combination with an effective amount of a compound of formula I 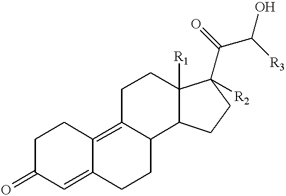
wherein R₁, R₂ and R₃ independently of each other are C₁₋₁₂alkyl, in the form of 21R or 21S epimers or mixtures thereof, or a pharmaceutically acceptable salt thereof, the effective amounts being sufficient to lower triglycerides.

3. A method of treating hyperlipoproteinemia, hypertriglyceridemia, hyperlipidemia or hypercholesterolemia, which method comprises administering to a subject an effective amount of an estrogen or estrogen receptor modulator in combination with an effective amount of a compound of formula I 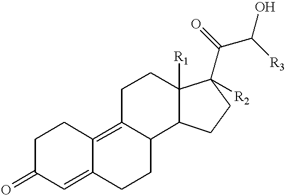
wherein R₁, R₂ and R₃ independently of each other are C₁₋₁₂alkyl, in the form of 21R or 21S epimers or mixtures thereof, or a pharmaceutically acceptable salt thereof, the effective amounts being sufficient to treat or prevent hyperlipidemia, hyperlipoproteinemia, hypertriglyceridemia or hypercholesterolemia.

4. A method of treating arteriosclerosis, which method comprises administering to a subject an effective amount of an estrogen or estrogen receptor modulator in combination with an effective amount of a compound of formula I 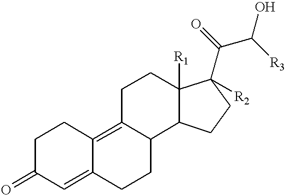
wherein R₁, R₂ and R₃ independently of each other are C₁₋₁₂alkyl, in the form of 21R or 21S epimers or mixtures thereof, or a pharmaceutically acceptable salt thereof, the effective amounts being sufficient to treat or prevent arteriosclerosis.

5. A method for the anticoagulative treatment of a patient comprising administering to the patient an effective amount of an estrogen or estrogen receptor modulator in combination with an effective amount of a compound of formula I 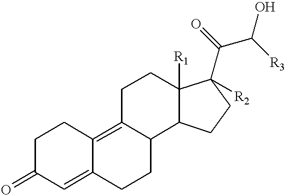
wherein R₁, R₂ and R₃ independently of each other are C₁₋₁₂alkyl, in the form of 21R or 21S epimers or mixtures thereof, or a pharmaceutically acceptable salt thereof, the effective amounts being sufficient to anticoagulate the patient.

6. The method of claim 3 wherein the estrogen or estrogen receptor modulator and the compound of formula I is administered simultaneously in one dosage form.

7. The method of claim 6, wherein the dosage form is a tablet, a capsule or a transdermal patch.

8. The method of claim 3 wherein the estrogen or estrogen receptor modulator and the compound of formula I is administered substantially simultaneously.

9. The method of claim 3 wherein the compound of formula I is

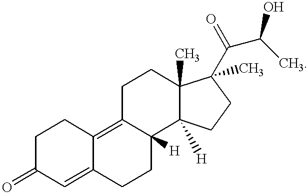

10. The method of claim 3 wherein the estrogen is selected from 17-beta-estradiol and esters thereof, ethinylestradiol, estriol (trihydroxyestrin), estrone, conjugated estrogens, sodium estrone sulfate, 8(9)-dehydroestradiol derivatives, 17alpha-dihydroequilin, equilenin, 17alpha-dihydroequilenin, esterified estrogens, and equilin.

11. The method of claim 3, wherein the effective amount of the estrogen or estrogen receptor modulator is from 0.0001 to 1000 mg/day and the effective amount of the compound of formula I is from 0.0001 to 1000 mg/day.

12. A kit containing a treatment for hyperlipoproteinemia, hypertriglyceridemia, hyperlipidemia or hypercholesterolemia or arteriosclerosis or for anticoagulative treatment, comprising: a) an effective amount of an estrogen or estrogen receptor modulator and a pharmaceutically acceptable carrier in a first unit dosage form; b) an effective amount of a compound of formula I 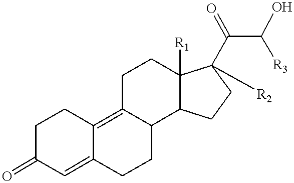
wherein R₁, R₂ and R₃ independently of each other are C₁₋₁₂alkyl, in the form of 21R or 21S epimers or mixtures thereof, or a pharmaceutically acceptable salt thereof, and a pharmaceutically acceptable carrier in a second unit dosage form; and c) means for containing the first and second dosage forms.

13. A pharmaceutical composition comprising an estrogen or estrogen receptor modulator and a compound of formula I 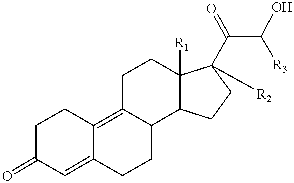
wherein R₁, R₂ and R₃ independently of each other are C₁₋₁₂alkyl, in the form of 21R or 21S epimers or mixtures thereof, or a pharmaceutically acceptable salt thereof, and a pharmaceutically acceptable carrier.

14. The pharmaceutical composition of claim 13, wherein the estrogen is 17-beta-estradiol and esters thereof or conjugated estrogens, and the compound of formula I is trimegestone.
